(19) United States
(12) Patent Application Publication
(10) Pub. No.: US 2002/0110583 A1
(43) Pub. Date: Aug. 15, 2002

(54) Hair cap with conditioner

(76) Inventor: Gary Grey, New York, NY (US)

Correspondence Address: OSTROLENK FABER GERB & SOFFEN, 1180 AVENUE OF THE AMERICAS, NEW YORK, NY, 100368403

(21) Appl. No.: 10/053,126

(22) Filed: Jan. 21, 2002

Related U.S. Application Data
(60) Provisional application No. 60/269,171, filed on Feb. 15, 2001.

(51) Int. Cl. A61K 7/075; A61K 7/08; A61K 9/70; B32B 27/12

(52) U.S. Cl. 424/443; 424/70.27; 424/70.31; 442/123

(57) ABSTRACT

A woven fabric having a sorbed hair conditioner is provided.

### CROSS REFERENCE TO RELATED APPLICATIONS

This application claims the benefit and priority of U.S. Provisional Application No. 60/269,171 filed Feb. 15, 2001 and entitled “HAIR COVERING WITH CONDITIONER”, and which is incorporated herein by reference.

### BACKGROUND OF THE INVENTION

Hair conditioning compositions are well known. They are intended to cause the hair to be soft, manageable and shiny. They also ease the ability to comb hair whether wet or dry, as well as preventing a condition known as “fly-away” of dry hair.

The application of most hair conditioning compositions is on wet hair, usually after the hair has been shampooed. More recently, so called two-in-one conditioning shampoos have been developed which allow a single application and provide both cleansing and conditioning actions in a single preparation.

Hair conditioners can generally be divided into two types. One is referred to in the art as “rinse off” because the conditioner is allowed to remain in contact with the hair for a brief period of time and is then removed by, for instance, rinsing. The other type is called a “leave on” product and this denotes a composition which is applied to the hair, whether in a wet or dry condition, and is not subsequently rinsed off. The “leave on” hair conditioners are typically applied to the hair from a pump-type spray dispenser from which the conditioner emerges either as a mist or a liquid stream.

Berryhill, et al. U.S. Pat. No. 3,088,469 discloses a hair conditioner and retainer with a rectangular swatch of a non-woven fabric that has lanolin applied thereto. The lanolin is preferably anhydrous and is applied to the non-woven by being divided into a fine mist in an ionization chamber, the particles of the mist being charged electrically and caused to precipitate by imposing an intense electrostatic field on the fabric.

There is a need for a hair covering with conditioner(s) which is easy to make and is also easy to use.

### SUMMARY OF THE INVENTION

The present invention provides a hair covering with conditioners which can be transferred to a person's hair. More particularly, a woven fabric having a sorbed hair conditioner is provided. The fabric is dry to the touch and does not appear to be wet or greasy. The hair conditioner can be easily desorbed from the fabric and transferred to the hair. Other features and advantages of the present invention will become apparent from the following description of the invention.

### DESCRIPTION OF THE INVENTION

In accordance with the present invention, a preferably knit fabric, preferably of a polyester or satin material having one or more sorbed hair conditioners is provided. The shape and style of the fabric is not restricted and it can be in the form of a bonnet type cap (similar to a shower cap) or a wrap which can be either tied or secured with hook and loop closures.

Any known hair conditioner or combination of conditioners can be employed in the present invention. It or they can be absorbed or adsorbed to the fabric by a variety of approaches. The only requirement of the sorbtion process is that it does not fix the conditioner to the fabric and allows the conditioner to be easily desorbed from the fabric and transferred to the hair.

One type of hair conditioner which can be used is the type containing a positive ionic charge. It is well known that adult human hair, especially hair in need of conditioning, carries a negative ionic charge and positively charged conditioner material is attracted to the negative sites on or in the hair and attach themselves thereto, thereby providing a conditioning effect. These cationic conditioning agents can be sorbed to a sleep cap, for example, and will come into contact with the hair while the person is sleeping.

A second type of hair conditioner is the heat activated conditioner. The heat which activates these materials can be the body's natural warmth or an applied heat from, for example, a blow dryer. The heat, combined with the friction of the hair on the treated fabric causes the conditioner(s) to deposit in or on the hair and provide a conditioning effect.

A third type of conditioner is a thixotropic material. These are conditioners which respond to warmth or body heat by becoming less viscous. As a result, it becomes easier to separate the conditioners from the fabric and transfer them to the hair by way of friction.

A fourth approach involves the use of a non-polar conditioner which is dissolved or suspended in a polar carrier such as an alcohol, water or a combination of water and alcohol, applied to the fabric and allowed to dry. This results in the deposit of a non-greasy hydrophobic material on and in the fabric. Friction between the fabric and the hair causes the transfer of the conditioning agents to the hair.

Another type of conditioner is a hydroscopic conditioning agent which has known humectant, emollient and film-forming properties. The hydroscopic nature of this type of conditioner works to retain moisture and also the moisture balance of the human hair. Transfer from the fabric to the hair is the result of friction with the natural moisture content of the human hair assisting in the transfer.

The hair covering of thepresent invention canbe easilyprepared. The following is an example of a process which can be employed.

A solution or mixture containing a combination of three conditioners, Dow Silicone 200/200, PEG 45 palm kernel glyceride and a hydrolyzed wheat protein with a silicone backbone (the latter being a heat-activated conditioner) is prepared using a combination of deionized water and isopropyl alcohol as a solvent, glycerine as a humectant, triethanolamine as an agent to adjust the pH to a value appropriate for contact with the hair as well as conventional anti-bacterial agents/preservatives, fragrance and anti-foaming agent, e.g., one obtained from Dow, Antifoam 1410.

The solution was transferred to a 100 denier single jersey knit polyester which had previously been knit, dyed and finished. The solution was sprayed on the fabric causing the aqueous solution of the conditioner to be coated on the fabric and causing it to become wet, i.e., slightly saturated with the mixture of conditioning agent and solvent.

The fabric was then re-rolled onto plastic cores.

The fabric was then placed in a well-ventilated chamber through which air was circulated at a rapid rate for 24 to 48 hours. The water and alcohol solvents evaporated and were removed from the chamber by being entrained in the air leaving the chamber, leaving the conditioning agents on and in the fabric. The resulting fabric containing the sorbed conditioners was dry to the touch.

It will be appreciated that various changes and modifications can be made to the product and process of this invention without departing from the spirit and scope thereof. The various embodiments which have been described herein were set forth in order to further illustrate the invention but were not intended to limit it.

What is claimed is:

1. A fabric having a sorbed hair conditioner, the fabric being dry to the touch.

2. The fabric of claim 1 wherein the fabric is a knitted fabric.

3. A fabric according to claim 2 in which the hair conditioner is a cationic hair conditioner.

4. A fabric according to claim 2 in which the hair conditioner is a heat-activated hair conditioner.

5. A fabric according to claim 2 in which the hair conditioner is a thixotropic hair conditioner.

6. A fabric according to claim 2 in which the hair conditioner is a non-polar hair conditioner.

7. A fabric according to claim 2 in which the hair conditioner is a hydroscopic hair conditioner.

8. A fabric according to claim 2 in which a plurality of the hair conditioners are sorbed.

9. A fabric according to claim 8 in the form of a wrap or bonnet for the hair.

10. A fabric according to claim 1 in the form of a wrap or bonnet for the hair.

11. A fabric according to claim 1, wherein the conditioner comprises a solution or mixture containing a combination of three conditioners, Dow Silicone 200/200, PEG 45 palm kernel glyceride and a hydrolyzed wheat protein with a silicone backbone (the latter being a heat-activated conditioner), a combination of deionized water and isopropyl alcohol as a solvent, glycerine as a humectant, triethanolamine as an agent to adjust the pH to a value appropriate for contact with the hair and antibacterial agents/preservatives, fragrance and anti-foaming agent.

12. A method of making the fabric having a sorbed conditioner, the fabric being dry to the touch, the method comprising sorbing a hair conditioner to a woven fabric and drying the resulting fabric until it is dry to the touch.

13. The method of claim 12 in which the woven fabric is a knitted fabric.

14. The method of claim 13 in which a solution of a hair conditioner is applied to the fabric followed by causing the solvent of the solution or dispersion to be evaporated and removed from the fabric.

15. The method of claim 14 in which the solution contains a combination of at least two hair conditioners.

16. The method of claim 15 in which at least one of the conditioners is heat activated.

17. The method of claim 12, wherein the conditioner comprises a solution or mixture containing a combination of three conditioners, Dow Silicone 200/200, PEG 45 palm kernel glyceride and a hydrolyzed wheat protein with a silicone backbone (the latter being a heat-activated conditioner), a combination of deionized water and isopropyl alcohol as a solvent, glycerine as a humectant, triethanolamine as an agent to adjust the pH to a value appropriate for contact with the hair and anti-bacterial agents/preservatives, fragrance and anti-foaming agent.
